(19) United States
(12) Patent Application Publication
(10) Pub. No.: US 2024/0425795 A1
(43) Pub. Date: Dec. 26, 2024

(54) BIOREACTOR WITH SENSOR ELEMENT

(71) Applicant: ORIBIOTECH LTD, London, Greater London (GB)

(72) Inventors: Farlan Veraitch, Greater London, London (GB); William Raimes, Greater London, London (GB); Stephen Shapka, London, Greater London (GB); Arman Amini, Greater London, London (GB); Jason Palmer, Melbourn, Hertfordshire (GB); Gary Kipling, Melbourn, Hertfordshire (GB); Lewis Roberts-James, Melbourn, Hertfordshire (GB)

(21) Appl. No.: 18/694,211

(22) PCT Filed: Sep. 22, 2022
(86) PCT No.: PCT/GB2022/052402

§ 371 Date: Mar. 21, 2024

(30) Foreign Application Priority Data
Sep. 24, 2021 (GB) 2113675.9

(51) Int. Cl. C12M 1/34; C12M 1/00; C12M 3/04

(52) U.S. Cl. CPC C12M 41/30; C12M 23/40; C12M 27/10

(57) ABSTRACT

The present disclosure provides a bioreactor for cell culturing. The bioreactor comprises a container comprising a base and a side wall defining an internal volume for holding a cell suspension. The container is rotatable during use about a rotational axis. The bioreactor also includes a sensor element disposed on an internal surface of the container for interaction with a sensor receiver positioned externally of the container and operable to interact with the sensor element to detect a characteristic of the cell suspension. The sensor element is offset from the rotational axis of the bioreactor and arranged to align with the sensor receiver in at least two rotational positions of the bioreactor during use.

### CROSS REFERENCE TO RELATED APPLICATIONS

This application is a national phase entry under 35 U.S.C. § 371 of International Patent Application PCT/GB2022/052402, filed Sep. 22, 2022, designating the United States of America and published as International Patent Publication WO 2023/047115 A1 on Mar. 30, 2023, which claims the benefit under Article 8 of the Patent Cooperation Treaty to Great Britain Patent Application Serial No. 2113675.9, filed Sep. 24, 2021.

### TECHNICAL FIELD

The present disclosure relates to a bioreactor, for example, a bioreactor for cell culturing.

### BACKGROUND

Cell and gene therapy manufacturing processes are often complex and include manual or semi-automated steps across several devices. Equipment systems used in various steps (i.e., unit operations) of cell-based therapeutic products (CTP) manufacturing may include devices for cell collection, cell isolation/selection, cell expansion, cell washing and volume reduction, cell storage and transportation. The unit operations can vary immensely based on the manufacturing model (i.e., autologous versus allogenic), cell type, intended purpose, among other factors. In addition, cells are “living” entities sensitive to even the simplest manipulations (such as differences in a cell transferring procedure). The role of cell manufacturing equipment in ensuring scalability and reproducibility is an important factor for cell and gene therapy manufacturing.

In addition, cell-based therapeutic products (CTP) have gained significant momentum thus there is a need for improved cell manufacturing equipment for various cell manufacturing procedures, for example, but not limited to stem cell enrichment, generation of chimeric antigen receptor (CAR) T cells, and various cell manufacturing processes such as collection, purification, gene modification, incubation/recovery, washing, infusion into patient and/or freezing.

The culture or processing of cells typically requires the use of a device to hold the cells, for example, in an appropriate culture medium when culturing the cells. The known devices include shaker flasks, roller bottles, T-flasks and bags.

A key limiting factor in the production of cells or gene therapies for use in medicine is the absence of compact, automated closed systems for performing unit operations without contamination. For example, during cell culture, upstream or subsequent processing of cells, there is a risk of contamination when making additions to the culture vessel, or when removing cells or removing liquid samples. The operating systems are largely manual and hence expensive to operate. Multiple pieces of equipment are typically required to cover all of the non-cell culture steps, which involves many transfers, each of which is an opportunity for operator errors and contamination to occur. Furthermore, with increasing manual operations comes increasing risk of manual errors and therefore the current labor-intensive processes lack the robustness required for the manufacture of clinical-grade therapeutics.

There is therefore a need for cell processing devices (e.g., multistep cell processors) that permit such processing that avoids the requirement for constant movement of cells into fresh devices.

### BRIEF SUMMARY

According to an aspect of the present disclosure, there is provided a bioreactor for cell culturing, the bioreactor comprising:
a container comprising a base and a side wall defining an internal volume for holding a cell suspension, the container being rotatable during use about a rotational axis; and
a sensor element disposed on an internal surface of the container for interaction with a sensor receiver positioned externally of the container and operable to interact with the sensor element to detect a characteristic of the cell suspension, and
wherein the sensor element is offset from the rotational axis of the bioreactor and arranged to align with the sensor receiver in at least two rotational positions of the bioreactor during use.

In examples, the sensor element is disposed on the base. In other examples, the sensor element is additionally or alternatively disposed on the side wall. The container, in particular, the base or side wall, may comprise a sensor window, and the sensor element may be disposed on the sensor window. The sensor window is preferably configured to permit transmission of light between the sensor element and the sensor receiver.

In examples, the rotational axis extends through the base and the internal volume of the container.

In examples, the sensor element comprises an annulus centered on the rotational axis of the bioreactor. Accordingly, the sensor receiver is aligned with the annulus sensor element regardless of the rotational position of the bioreactor. In examples where the sensor element is on the base, the sensor element forms an annulus on the base. In examples where the sensor element is on the side wall, the sensor element extends about the circumference of the side wall.

In examples, the sensor element may comprise a plurality of discrete portions. The discrete portions may be arranged on the base, in particular, on a common circumference from the rotational axis, or on the side wall.

In examples, the sensor element or at least one of the discrete portions of the sensor element may comprise an annular sector. The annular sector may extend about the rotational axis by between about 20 degrees and about 350 degrees so that the sensor receiver is aligned with the annular sector sensor element through a portion of the rotation of the bioreactor. In examples, the annular sector may extend about the rotational axis by any amount, for example, between about 20 degrees and about 180 degrees, or between about 45 degrees and about 90degrees.

In examples, the sensor element or at least one of the discrete portions of the sensor element may comprise a dot, for example, a circular dot or square dot. The dots may be evenly distributed about the rotational axis, for example, twelve dots can be positioned at 30degree intervals, four dots can be positioned at 90 degree intervals, or three dots can be positioned at 120 degree intervals, etc.

In examples, the bioreactor may comprise a second sensor element disposed on an internal surface of the container. In examples, the second sensor element may be disposed on the sensor window, or on a second sensor window. The second sensor element may be aligned with the rotational axis of the bioreactor and arranged to align with a second sensor receiver positioned externally of the base and operable to interact with the second sensor element to detect a characteristic of the cell suspension. Accordingly, the second sensor receiver and second sensor element are aligned regardless of the rotational position of the bioreactor. In examples, the sensor element may be spaced from the second sensor element, or they may be abutting along an edge.

In examples, the bioreactor may comprise a third sensor element disposed on an internal surface of the container. In examples, the third sensor element may be disposed on the sensor window, on the second sensor window, or on a third sensor window. The third sensor element may be offset from the rotational axis of the bioreactor and arranged to align with a third sensor receiver positioned externally of the base and operable to interact with the third sensor element to detect a characteristic of the cell suspension. In examples, the third sensor element is arranged to align with the third sensor receiver in at least two rotational positions of the bioreactor during use. In examples, the sensor element may be spaced from the third sensor element, or they may be abutting along an edge.

In examples, the sensor element and/or second sensor element may be adhered to the internal surface of the container, for example, to an internal surface of the sensor window. In examples, the sensor element and/or the second sensor element comprises an oxygen-sensitive coating or a pH-sensitive coating. In examples, the sensor element and/or the second sensor element may comprise a substrate having an oxygen-sensitive coating or a pH-sensitive coating on one side and an adhesive on the other side for fixing to the sensor window. The sensor element and/or second sensor element, in particular, a material or coating of the sensor element and/or second sensor element, may be configured to respond to incident light that induces a fluorescent signal based on a characteristic of the cell suspension within the bioreactor, for example, an oxygen concentration or pH of the cell suspension. Accordingly, the sensor receiver can receive the fluorescent signal and a sensor meter can be used to measure the fluorescent signal to determine the characteristic of the cell suspension.

The sensor window is transparent or translucent to the wavelength of light at which the sensor element and sensor receiver operate. In examples, the sensor window comprises a transparent or translucent material, for example, a polymer such as polycarbonate, polyethylene terephthalate, or polymethyl methacrylate, or another material such as glass.

In examples, the side wall comprises a compressible side wall, for example, a bellows wall. Accordingly, the container may be a compressible or deformable container.

In examples, the bioreactor may further comprise an interface plate attached to the side wall opposite to the base. The interface plate may comprise one or more ports for adding fluid to, or extracting fluid from, the container.

According to a further aspect of the present disclosure, there is also provided a bioreactor system comprising the bioreactor described above, and a housing adapted to support the bioreactor such that the bioreactor is rotatable relative to the housing about the rotational axis.

In examples, the housing may be an incubator housing configured to control an environment of the bioreactor.

For example, the bioreactor may comprise an interface plate attached to the side wall opposite to the base, and the housing may comprise a bioreactor receiving portion adapted to support the interface plate such that the side wall and base are suspended below the interface plate.

The interface plate may comprise a plurality of connector interfaces for connecting a consumable for adding fluid to, or extracting fluid from, the bioreactor. The plurality of connector interfaces may be spaced about the interface plate at a common distance from the rotational axis of the bioreactor. The bioreactor may be rotatable to index between connector interfaces on the interface plate. For example, the bioreactor system may include a consumable attachment point for attaching a consumable to the bioreactor system such that it can engage a connector interface on the interface plate. The bioreactor can be rotated to bring different connecter interfaces into alignment with the consumable attachment point. In examples, the sensor element and the sensor receiver are arranged to be aligned with each other in each of the rotational positions of the bioreactor that correspond to alignment between a connector interface and the consumable attachment point.

In examples, the housing, in particular, the bioreactor receiving portion, may comprise an actuator operable to rotate the bioreactor.

In examples, the bioreactor system may further comprise a sensor receiver positionable externally of the base and arranged to be aligned with the sensor element in at least two rotational positions of the bioreactor relative to the housing.

In examples, the sensor receiver may comprise an optical receiver, for example, an optical fiber. The bioreactor system may further comprise a sensor meter arranged to receive an optical signal from the optical receiver. In examples, the sensor receiver includes an optical mount arranged to position the optical receiver, in particular, the optical fiber, in alignment with the sensor element. In examples, the sensor meter may be configured to transmit a sensor excitation signal to the sensor element, and to receive a sensor signal returned from the sensor element.

In examples, the sensor receiver may be movably mounted and movable to an operational position in which the sensor receiver is in contact with, or adjacent to, the container of the bioreactor proximal to the sensor element. In some examples, in the operational position the sensor receiver is in contact with, or adjacent to, a surface of the base or side wall of the container, for example, a sensor window. Specifically, in the operational position the sensor receiver may abut, or be proximal to, the sensor window. For example, the sensor receiver may be positioned within about 10 millimeters of the external surface of the container (e.g., sensor window), preferably within about 7 millimeters.

In examples, the bioreactor system may include a second sensor receiver for a second sensor element, as described above. In such examples, the second sensor receiver may be mounted in the same manner as the sensor receiver described above. The sensor receiver and second sensor receiver may be provided in a sensor unit that is movable to the operational position.

In examples, the bioreactor system may comprise an actuator operable to move the sensor receiver and/or the sensor unit into the operational position.

In examples, the side wall of the bioreactor may comprise a compressible side wall, for example, a bellows wall. In such examples, the bioreactor system may further comprise an agitator operable to engage the base of the bioreactor. The agitator may be operable to compress the container of the bioreactor, and/or to tilt the base of the bioreactor, in order to agitate the contents of the bioreactor.

In examples, the sensor receiver may be mounted to the agitator such that the sensor receiver and/or sensor unit is in the operational position when the agitator engages the base of the bioreactor. For example, the agitator may comprise an agitator plate operable to engage the base of the bioreactor, and the sensor receiver and/or sensor unit may be mounted to the agitator plate.

In examples, the agitator may be configured to couple to the base of the bioreactor. In particular, an agitator plate of the agitator may be configured to couple to the base, for example, by a mechanical clip or by an electromagnetic coupling.

In examples, the agitator may be configured to decouple from the base to permit rotation of the bioreactor relative to the housing. Alternatively, the agitator, in particular, the agitator plate, may include a rotatable portion that is rotatable with the bioreactor.

According to a further aspect of the present disclosure, there is also provided a method of culturing cells in the bioreactor system described above, the method comprising:
loading the bioreactor into the housing;
providing a cell suspension in the container of the bioreactor;
rotating the bioreactor; and
sensing a characteristic of the cell suspension by the sensor element and sensor receiver in at least two rotational positions of the bioreactor.

As described above, the bioreactor may be rotated in order to bring different connector interfaces of the bioreactor into alignment with a consumable attachment point on the housing.

In examples, the method may further comprise agitating the cell suspension in the container.

In examples, sensing a characteristic of the cell suspension may comprise sensing a dissolved oxygen or pH of the cell suspension.

When sensing the characteristic of the cell suspension an optical signal may pass from the sensor element to the sensor receiver.

### BRIEF DESCRIPTION OF THE DRAWINGS

Embodiments of the invention are further described hereinafter with reference to the accompanying drawings, in which:

FIG. 1 shows a bioreactor having a cell culture container;

FIGS. 2A to 2D show a housing assembly with a bioreactor and consumable being loaded into the housing assembly;

FIG. 3 shows an agitator of the housing assembly in a first position;

FIG. 4 shows the agitator in a second position;

FIG. 5 shows an actuator arrangement for the agitator;

FIGS. 6A to 6C show different agitation movements of the agitator;

FIG. 7 shows the agitator engaged with the bioreactor, and a sensor assembly;

FIG. 8 shows a cross-section of the base of the bioreactor and the sensor assembly; and

FIGS. 9A to 9D show different arrangements of the sensor elements within the bioreactor.

### DETAILED DESCRIPTION

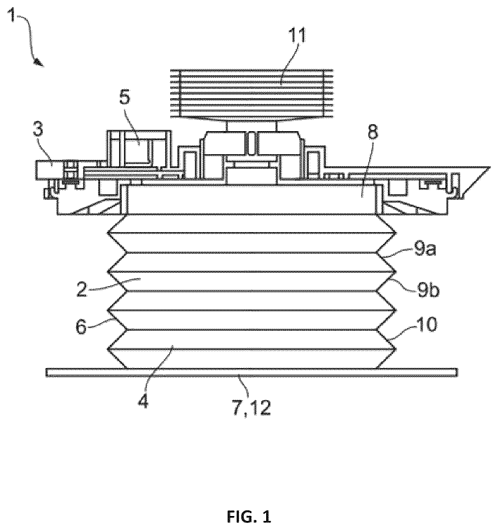

The bioreactor 1 shown in FIG. 1 includes a cell culture container 2 and an interface plate 3. During use the cell culture container 2 holds a fluid 4 in which the cell processing occurs. In particular, the fluid 4 is a cell suspension comprising a population of cells present in a liquid medium. The cells are cultured to reproduce, and may be otherwise treated, to create a cell-based therapeutic product.

The interface plate 3 is attached to a top of the cell culture container 2, for example, acting as a lid or closure. The interface plate 3 comprises at least one connector interface 5 for connecting to an external component, for example, a consumable for delivering a fluid to, or extracting a fluid from, the cell culture container 2. Preferably, the interface plate 3 includes a plurality of connector interfaces 5 for connecting to external components. Each connector interface 5 may be used once or more for adding or removing fluid. The connector interfaces 5 may be distributed about the interface plate 3. Accordingly, the interface plate 3 provides for adding media and other fluids to the cell culture container 2 during cell processing, and/or for removing fluid from the cell culture container 2 during processing, for example, to remove a sample or waste fluid.

The cell culture container 2 may be extendable and/or compressible. In particular, the cell culture container 2 has a compressible wall element 6, for example, a bellows wall. The cell culture container 2 has a base 7 disposed opposite to the interface plate 3, and a compressible wall element 6 defining a sidewall of the cell culture container 2. A top part of the compressible wall element 6 is attached to the interface plate 3. The top part of the compressible wall element 6 may include a rigid ring 8 or similar for attaching to the interface plate 3. The compressible wall element 6 is compressible and/or extendible such that the base 7 can move toward and away from the interface plate 3, changing the internal volume of the cell culture container 2. The base 7 may be moved relative to the interface plate 3 in order to agitate or mix the fluid 4 in the cell culture container 2.

The compressible wall element 6 may be a bellows wall, having a concertina arrangement that allows the compressible wall element 6 to fold onto itself in order to compress. In particular, as illustrated the compressible wall element 6 may comprise a series of alternately arranged deformable portions 9a, 9b, specifically inwardly deformable portions 9a and outwardly deformable portions 9b. Leaf segments 10 extend between the deformable portions 9a, 9b. The leaf segments 10 are more rigid than the deformable portions 9a, 9b. The deformable portions 9a, 9b act as hinges that allow the compressible wall element 6 to collapse like a bellows or concertina, with the leaf segments 10 remaining substantially non-deformed.

The compressible wall element 6 may comprise at least one inwardly deformable portion 9a and at least one outwardly deformable portion 9b, for example, at least two inwardly deformable portions 9a and at least two outwardly deformable portions 9b. The compressible wall element 6 may comprise three, four, or more inwardly deformable portions 9a and three, four or more outwardly deformable portions 9b.

The inwardly deformable portion(s) 9a and outwardly deformable portion(s) 9b may be formed by thinned sections in the compressible wall element 6. The inwardly deformable portion(s) 9a may comprise a thinned section arranged on the outer surface of the compressible wall element 6 such that it is deformable in an inwards direction. The outwardly deformable portion(s) 9b may comprise a thinned section arranged on the inner surface of the compressible wall element 6 such that it is deformable in an outwards direction.

In examples, the compressible wall element 6 comprises a silicone, in particular, a liquid silicone rubber. In other examples, the compressible wall element 6 comprises a low density polyethylene (LDPE). In other examples, the compressible wall element 6 comprises a thermoplastic elastomer (TPE). In examples, as described further hereinafter, the compressible wall element 6 may be coated, laminated, or otherwise treated to reduce the gas permeability of the compressible wall element 6 or to render the compressible wall element 6 impermeable to gases, particularly oxygen. In some examples, the compressible wall element 6 comprises a layer and an outer sheath, jacket, or coating. For example, the compressible wall element 6 may comprise an inner portion and a jacket over-molded onto the LDPE inner portion. The inner portion may comprise LDPE and the jacket may comprise a TPE. In another example, the compressible wall element 6 may comprise an elastomer outer, for example, a TPE outer, and a liner. For example, an LDPE liner may be blow-mounted onto the internal surface of the elastomer outer to form the liner. In another example, the liner may be an insert, for example, an LDPE insert, received within the elastomer outer but not co-molded with the elastomer outer. In such an example, it may be preferable that the liner comprises a base sheet and defines a sealed container (except for the top) to hold the cell culture.

The cell culture container 2 can therefore expand and contract, or be expanded and contracted, according to the material held in the cell culture container 2. In particular, the cell culture container 2 may expand as the volume of fluid 4 within the cell culture container 2 grows, and/or as additional materials are added.

As illustrated, the interface plate 3 also includes an expansion container 11, otherwise called a breathing container. The expansion container 11 allows for the cell culture container 2 to expand and contract without greatly changing the pressure in the cell culture container 2. Alternatively or additionally, the expansion container 11 may be operable, for example, by being mechanically or manually compressed or expanded, to expand or retract the compressible wall 6 of the cell culture container 2 and thereby change a volume of the cell culture container 2. Alternatively or additionally, the expansion container 11 may be operable, for example, by being mechanically or manually compressed or expanded, to alter the pressure within the cell culture container 2.

In various examples, the base 7 comprises a rigid base plate 40. The rigid base plate 40 is generally planar, i.e., flat. The rigid base plate 40 is attached to, or molded with, the compressible wall element 6.

The rigid base plate 40 is substantially planar and thereby defines a rigid, substantially flat bottom of the cell culture container 2. A flat bottom of the cell culture container 2 may provide for improved cell culturing, in particular, mixing and control over cell culturing. The flat bottom of the cell culture container 2 helps to ensure that cells are substantially evenly spread over the cross-section of the cell culture container 2 as the cells will sink to the bottom of the cell culture container and if the base 7 were not flat the cells would therefore be concentrated in a smaller volume, which may be detrimental to cell culturing. The flat bottom of the cell culture container 2 also helps to prevent fluid 4 being trapped in the cell culture container 2 when the cells are harvested or extracted at the end of the cell culturing process.

In various examples, the rigid base plate 40 comprises a thermoplastic, for example, a high density polyethylene (HDPE), or a polycarbonate (PC), or another rigid polymer. As described further hereinafter, the rigid base plate 40 may be opaque, transparent, or translucent.

In various examples, described in more detail hereinafter, the base 7, in particular, the rigid base plate 40, has a sensor window. The sensor window is transparent or translucent and provides an optical path into the cell culture container. Accordingly, a sensor can be arranged at least partially outside of the cell culture container and light can be transmitted through the sensor window in order to sense a characteristic of the cell culture within the cell culture container.

In the illustrated examples, the cell culture container 2 is generally cylindrical, with a generally circular base 7 and a generally cylindrical compressible wall element 6. Accordingly, an axial direction is defined between the base 7 and the end of the compressible wall element 6 where the interface plate 3 is mounted. However, it will be appreciated that the cell culture container 2 may take an alternative form, such as having a generally triangular or square cross-sectional form.

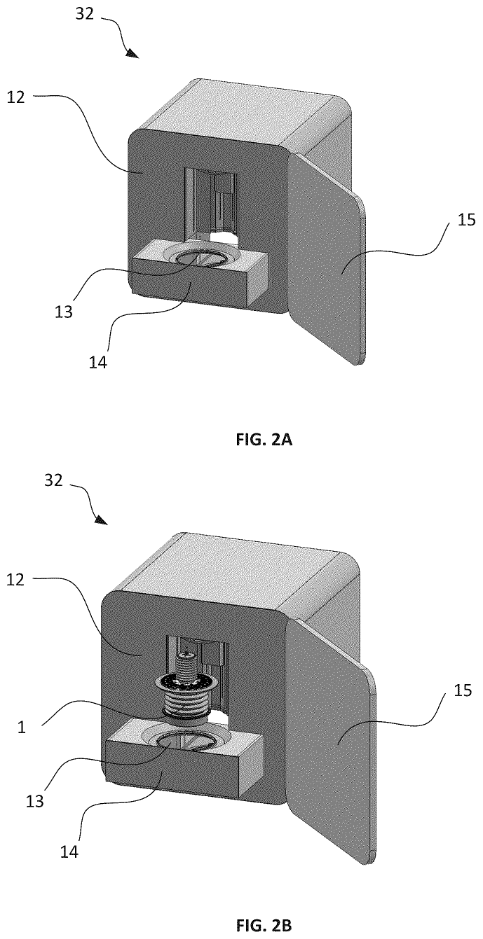

FIGS. 2A to 2D illustrate a bioreactor system 32 that includes a housing 12 and the bioreactor 1 described above. As shown in FIGS. 2A and 2B, in use, the bioreactor 1 is loaded into the housing, which may be an incubator housing 12. The incubator housing 12 may provide a controlled environment for the bioreactor 1. For example, the incubator housing 12 may control the temperature and gas concentrations of the air within the incubator housing 12 and surrounding the bioreactor 1 so as to control environmental aspects of the bioreactor 1. For example, the incubator housing 12 may include a heating and/or cooling unit that maintains the temperature within the incubator housing 12, preferably at about 37 degrees Celsius. The incubator housing 12 may additionally or alternatively include means for varying oxygen and carbon dioxide concentrations of the air within the incubator housing 12.

As shown in FIGS. 2A and 2B, the incubator housing 12 includes a receiving portion 13 to receive the bioreactor 1. In this example, the receiving portion 13 is formed in a drawer 14 for ease of loading and unloading the bioreactor 1 into the bioreactor system 32. The drawer 14 can be pulled out, the bioreactor 1 loaded into the receiving portion 13, and then the drawer 14 can be pushed into the incubator housing 12. A door 15 can be closed to seal the incubator housing 12. The receiving portion 13 can support the interface plate (3, see FIG. 1) of the bioreactor 1, and the container (2, see FIG. 1) can be suspended below the interface plate (3, see FIG. 1).

As described above, the interface plate 3 of the bioreactor includes a number of connector interfaces 5 for accessing the cell culture container in a sterile manner. In examples, the connector interfaces 5 are distributed about the interface plate 3 in a circle, so that each connector interface 5 is equally radially spaced from a central axis of the bioreactor 1. In examples, bioreactor system 32 includes a consumable attachment point 16, as illustrated in FIGS. 2C and 2D, for attachment of a consumable 17 to the bioreactor system 32 for engaging a connector interface 5 of the bioreactor 1. As shown, the consumable 17 is connectable to the consumable attachment point 16 in a generally vertical orientation, with a connector portion connected to the consumable attachment point 16. Once the consumable 17 has been connected to the consumable attachment point 16 a fluid path can be created through the interface plate 3 and into the cell culture container. The fluid path can be created via a sterile connector that may include a needle arranged to pierce a septum seal to create the fluid path. Such a sterile connector is described, for example, in WO2021/123760A1, which is hereby fully incorporated by reference.

In examples, the consumable 17 may be used to add material to, or extract material from, the bioreactor via the connector interface 5. The material may be a fluid, for example, a cell suspension, a cell culturing media, a virus suspension, or the like.

Referring to FIGS. 1 to 2D, the bioreactor 1 is rotatable within the incubator housing 12. In particular, the bioreactor 1 is rotatable within the receiving portion 13. When the bioreactor 1 is loaded into the receiving portion 13, the interface plate 3 is substantially horizontal, and the bioreactor 1 is rotatable about an axis extending substantially vertically with respect to the interface plate 3. The axis of rotation of the bioreactor 1 may extend through a center of the interface plate 3, and through the cell culture container and the base 7. In the illustrated examples, the bioreactor 1 is generally cylindrical and the interface plate 3 is generally circular, and the rotational axis is central to the bioreactor 1 and interface plate 3. The interface plate 3 is supported on the receiving portion 13 and the container 2 suspended below. The bioreactor 1 may be manually rotated by moving the interface plate 3, or the bioreactor system 32 (e.g., the receiving portion 13) may include an actuator (e.g., electric motor) for rotating the interface plate 3 and bioreactor 1 within the receiving portion 13.

In use, the bioreactor 1 is rotatable to align different connector interfaces 5 with the consumable attachment point 16. Accordingly, different connector interfaces 5 can be brought into alignment with the consumable attachment point 16 by rotation of the bioreactor 1 within the incubator housing 12. In this way, a single consumable attachment point 16 can be provided, and different connector interfaces 5 are selected by rotation of the bioreactor 1. In examples, each connector interface 5 on the interface plate 3 is used only once. To add to or remove a fluid from the bioreactor 1 the bioreactor 1 is rotated to bring a connector interface 5 into alignment with the consumable attachment point 16, the consumable 17 is attached, operated, and then detached, and then the bioreactor 1 can be rotated to bring a further connector interface 5 into alignment with the consumable attachment point 16 for a further fluid addition or removal operation.

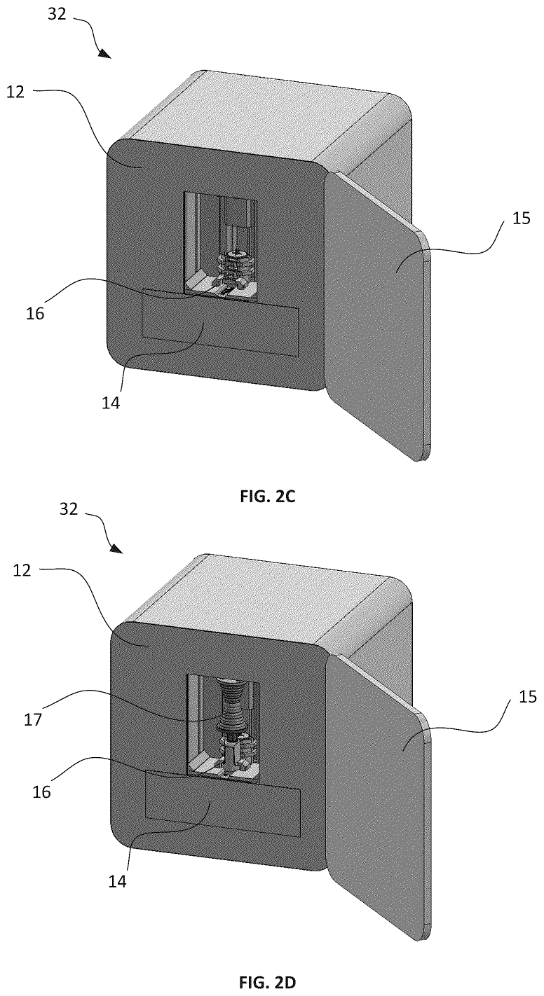

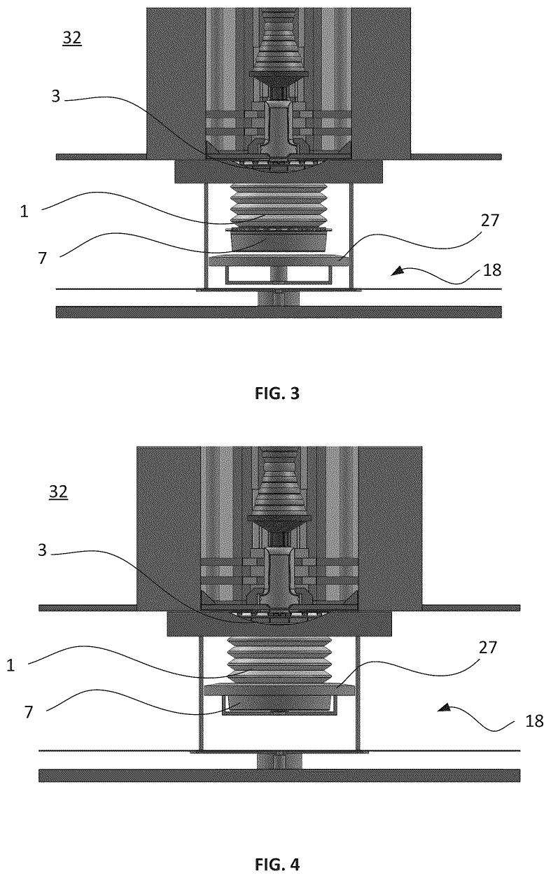

As shown in FIGS. 3 and 4, the bioreactor system 32 also includes an agitator 18. The agitator 18 is mounted within the incubator housing 12 and engages the base 7 of the bioreactor 1 to move the base 7 relative to the interface plate 3 and thereby agitate the contents of the bioreactor 1. Such agitation can help the cell culturing process, for example, by mixing fluids within the bioreactor 1, or by encouraging oxygen to dissolve into the cell culture.

As shown in FIG. 3, the agitator 18 includes an agitator plate 27 that is positionable so as to be spaced from the base 7 of the bioreactor 1, for example, during loading of the bioreactor 1 into the incubator housing 12. As shown in FIG. 4, the agitator 18 is operable to move the agitator plate 27 into engagement with the base 7 of the bioreactor 1. In the position shown in FIGS. 4, the agitator plate 27 may couple to the base 7 of the bioreactor 1, or may abut the base 7 of the bioreactor 1. Coupling of the agitator plate 27 to the base 7 may provide for improved control of the agitation of the bioreactor 1. Alternatively, if the agitator plate 27 is not coupled to the base 7, then impact contacts between the agitator plate 27 and the base 7 may provide desirable agitation of the contents of the bioreactor 1.

In examples, the agitator plate 27 may couple to the base 7 by a clip, or the agitator plate 27 may include an electromagnet and the base 7 may include a ferromagnetic portion so that the electromagnet is operable to couple the agitator plate 27 to the base 7.

In examples where the agitator plate 27 couples to the base 7 of the bioreactor 1, the coupling may permit rotation of the bioreactor 1 relative to the agitator 18. For example, a part of the agitator plate 27 may be rotatable with the base 7. In other examples, the agitator plate 27 may be decoupled from the base 7 to permit rotation of the bioreactor 1 relative to the agitator 18.

The agitator 18 comprises an actuator to move the agitator plate 27 relative to the bioreactor 1 within the incubator housing 12. The actuator moves the agitator plate 27 in an agitative movement.

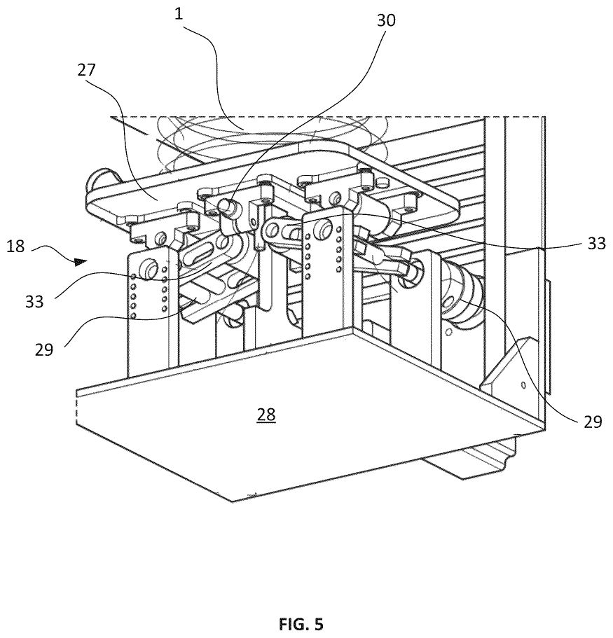

FIG. 5 illustrates an example agitator 18 with an actuator mechanism arranged to move the agitator plate 27. As shown, the agitator plate 27 is moveable to engage the bioreactor 1, in particular, the base (7, see FIG. 1). The actuator mechanism is mounted on a base plate 28. Between the base plate 28 and the agitator plate 27 is one or more actuators 29 that act to raise and lower the agitator plate 27.

In the illustrated example, the actuators 29 are motors arranged to rotate articulated crank arms 33 that are rotatably connected to the base plate 28 and to the agitator plate 27 such that rotation of the articulated crank arms 33 moves the agitator plate 27. In other examples, the linear actuators may be provided to act directly between the base plate 28 and the agitator plate 27.

Supports and guides may guide the movement of the agitator plate 27.

The actuator mechanism may further include a pivotable rod 30 such that the agitator plate 27 can pivot about the pivotable rod 30 to tilt the base of the bioreactor 1. Pivoting can be provided by raising one linear actuator 29 by a different amount to the other. Accordingly, the agitator plate 27 can be moved relative to the base plate 28 in order to engage the base of the bioreactor 1 and agitate the contents of the bioreactor 1.

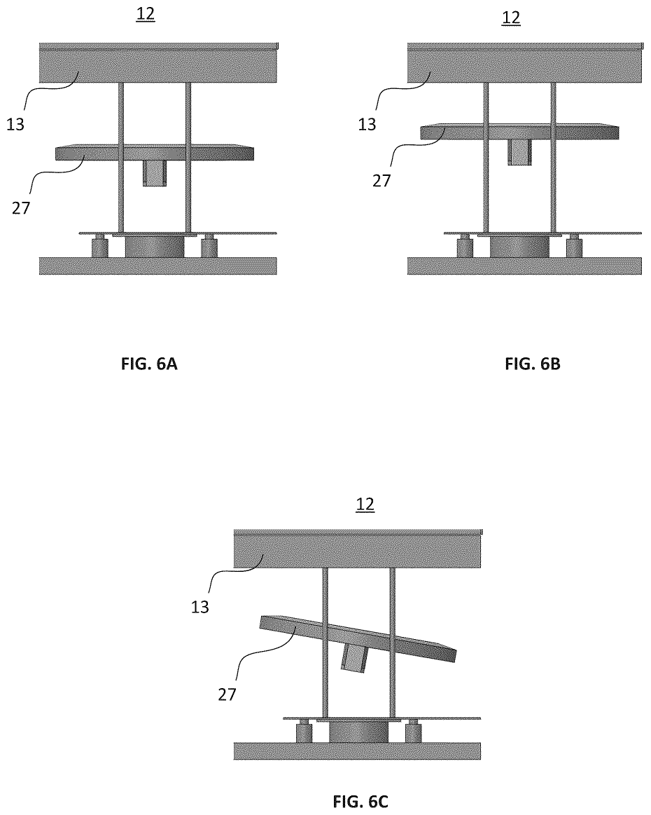

FIGS. 6A to 6C illustrate agitative movements of the agitator plate 27 without the bioreactor 1 or actuator mechanism shown. FIGS. 6A to 6C show the receiving portion 13 of the incubator housing 12, which supports the interface plate 3 of the bioreactor 1 during use as shown in FIGS. 2A and 3B, with the cell culture container (2, see FIG. 1) suspended below the receiving portion 13. As shown in FIGS. 6A and 6B, the agitative movement may be movement of the agitator plate 27 between lower and higher positions, possibly reciprocal, to agitate the contents of the bioreactor. In the example of FIG. 6C, the agitator plate 27 may tilt to agitate the contents of the bioreactor. A combination of vertical and tilting movements may be provided to agitate the contents of the bioreactor 1. The agitator plate 27 may be tiltable in different directions, and/or the agitator plate 27 may only be tiltable in one or two directions but rotation of the bioreactor 1 can change the direction of tilt of the bioreactor 1 itself.

Referring to FIGS. 3 to 6C, it will be appreciated that in examples where the agitator plate 27 couples to the base 7 of the bioreactor 1 then the base 7 will move with the agitator plate 27, and in examples where the agitator plate 27 does not couple to the base 7, the base 7 may lift wholly or partially away from the agitator plate 27 in some positions and/or during some agitative movements. Some agitative movements may provide impact contacts between the agitator plate 27 and the base 7 in order to agitate the contents of the bioreactor. In other examples, the agitator plate 27 may be vibratable to vibrate the base 7 of the bioreactor 1.

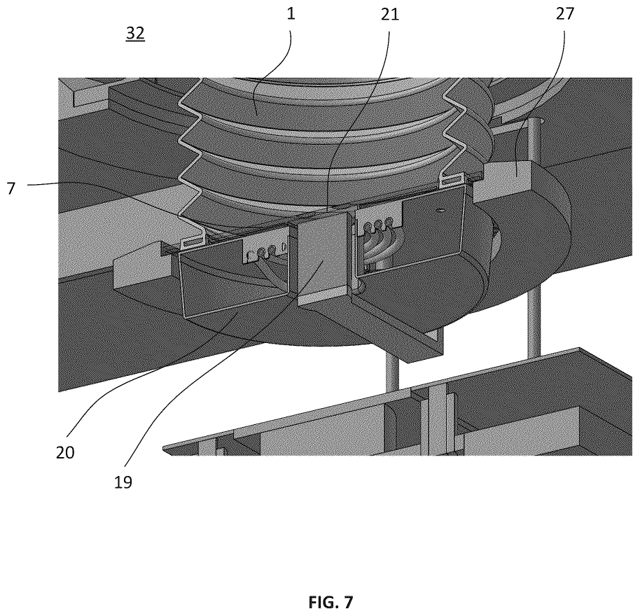

As shown in FIG. 7, the bioreactor system 32 includes a sensor unit 19 positionable adjacent to, or in contact with, the base 7 of the bioreactor 1. In particular, the sensor unit 19 is positionable adjacent to, or in contact with, a sensor window 21 formed in the base 7. For example, the sensor unit 19 is positionable within about 10 millimeters of the surface of the sensor window 21, preferably within about 7 millimeters of the surface of the sensor window 21. The sensor unit 19 may include a housing 20 as illustrated.

The sensor window 21 is transparent or translucent to a wavelength of light at which the sensor unit 19 operates. That is, the sensor window 21 permits transmission of light from within the bioreactor 1 to the sensor unit 19 on the exterior of the bioreactor 1. In some examples, the base 7 is formed of a transparent or translucent material (e.g., polycarbonate), in which example the sensor window 21 is a portion of the base 7. In other examples, the sensor window 21 is a transparent or translucent insert provided in a portion of the base 7.

In examples, the sensor unit 19 is provided within the incubator housing 12. In examples, the sensor unit 19 comprises an actuator that moves the sensor unit 19 into its operational position adjacent to, or in contact with, the base 7. The actuator may move the sensor unit 19 toward and away from the bioreactor 1 (in the direction of the rotational axis of the bioreactor 1), and may optionally tilt the sensor unit 19 to match the base 7 if the base 7 is also tilted. The actuator may be similar to the actuator mechanism of the agitator 18 as described with reference to FIG. 5.

In some examples, the sensor unit 19 may be mounted to the agitator 18, in particular, the agitator plate 27. In these examples, the sensor unit 19 will be in the operational position relative to the base 7 when the agitator plate 27 is engaged with the base 7 of the bioreactor 1. In particular, the sensor unit 19 may be mounted to the agitator plate 27 such that when the agitator plate 27 is engaged with the base 7 of the bioreactor 1 the sensor unit 19 is in contact with the base 7 or adjacent to the base 7, for example, within about 10 millimeters.

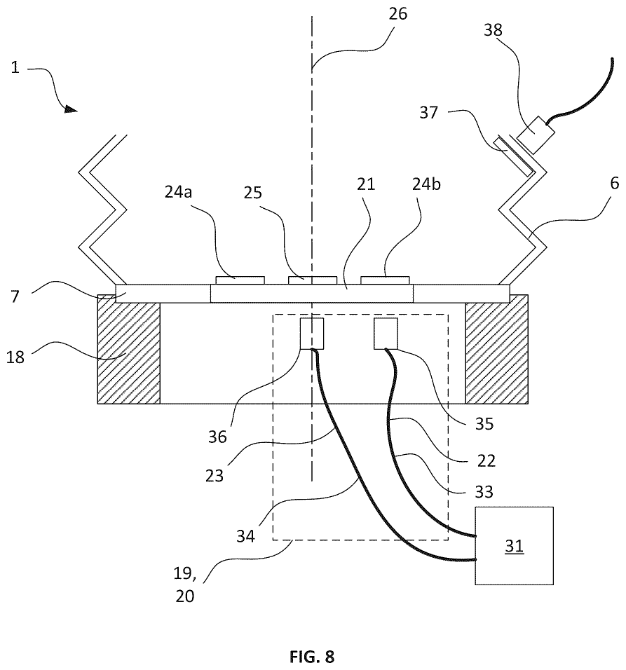

FIG. 8 schematically illustrates the sensor unit 19 in the operational position relative to the base 7 of the bioreactor 1. As illustrated, the sensor unit 19 includes a first sensor receiver 22 and a second sensor receiver 23. Within the bioreactor 1, on the inside surface of the sensor window 21 in the base 7, is a first sensor element 24 and a second sensor element 25. The first sensor receiver 22 is aligned with the first sensor element 24, and the second sensor receiver 23 is aligned with the second sensor element 25, as described in more detail hereinafter.

As mentioned above, the sensor window 21 is transparent or translucent, and in particular, transparent or translucent to the wavelength of light at which the sensor receivers 22, 23 and sensor elements 24, 25 operate. Accordingly, optical sensor signals can pass between the sensor receivers 22, 23 and sensor elements 24, 25, through the sensor window 21.

The sensor elements 24, 25 comprise a characteristic-sensitive material, for example, an oxygen-sensitive or pH-sensitive material, possibly as a coating. The optical properties of the sensor elements 24, 25 thereby change according to the corresponding characteristic of the cell suspension within the bioreactor 1.

The sensor receivers 22, 23 may include optical receivers, in particular, optical fibers 34, 39, configured to transmit light from an external sensor meter 31 through the sensor window 21 to the sensor elements 24, 25, and to transmit light from the sensor elements 24, 25 through the sensor window 21 to the external sensor meter 31. The optical fibers 34, 39 may be mounted to the sensor unit 19 at optical mounts 35, 36 that position the optical fibers 34, 39 toward the corresponding sensor elements 24, 25 so that light can be directed from the optical fibers 34, 39 onto the sensor elements 24, 25, and light can be received by the optical fibers 34, 39 from the sensor elements 24, 25.

Accordingly, the sensor meter 31 and sensor receivers 22, 23 are operable to direct light onto the sensor elements 24, 25 and to detect light from the sensor elements 24, 25. The detected light is indicative of the corresponding characteristic of the cell suspension in the bioreactor 1, in particular, dissolved oxygen and/or pH according to the configuration of the sensor elements 24, 25.

As shown in FIG. 8, the first sensor receiver 22, in particular, the first optical mount 35, and the first sensor element 24 are offset from the rotational axis 26 of the bioreactor 1. The first sensor element 24 is provided in at least two positions on the sensor window 21 that correspond to the offset of the first sensor receiver 22, in particular, the first optical mount 35. In the cross-section shown in FIG. 8 the first sensor element 24 has a first portion 24a aligned with the first sensor receiver 22 and a second portion 24b, diametrically opposite the first portion 24a, that would be aligned with the first sensor receiver 22 if the bioreactor 1 were rotated by 180 degrees relative to the sensor unit 19. Accordingly, the first sensor receiver 22 will be aligned with a portion of the first sensor element 24 in at least two rotational positions of the bioreactor 1.

In the example of FIG. 8, the second sensor receiver 23 and the second sensor element 25, in particular, the second optical mount 36, are aligned with the rotational axis 26 of the bioreactor 1. In this way, the second sensor receiver 23 and the second sensor element 25 remain aligned regardless of the rotational position of the bioreactor 1. Accordingly, sensor readings can be obtained when the bioreactor 1 has been rotated.

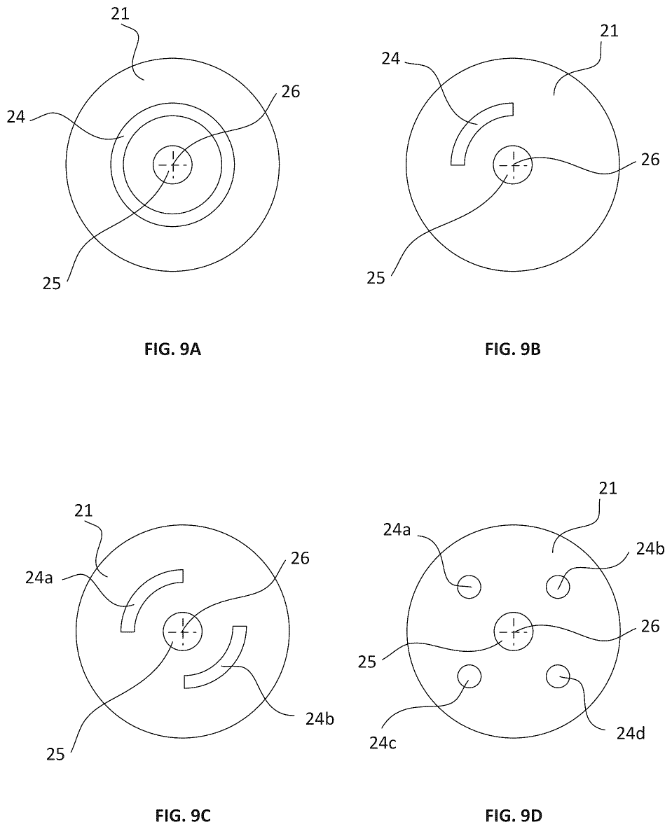

FIGS. 9A to 9D illustrate different examples of the arrangement of the first and second sensor elements 24, 25 on the sensor window 21 in the bioreactor. In each of these examples, the second sensor element 25 is aligned with the rotational axis 26 of the bioreactor, in this example, centrally within the sensor window 21. Accordingly, the second sensor receiver (23, see FIG. 8) will be aligned with the second sensor element 25 regardless of the rotational position of the bioreactor (1, see FIG. 8). In the illustrated examples, the second sensor element 25 is circular, for example, a sensor element dot, but in other examples, the second sensor element 25 may have a different shape, for example, square.

In FIG. 9A, the first sensor element 24 comprises an annulus centered on the rotational axis 26 of the bioreactor 1 such that the annular first sensor element 24 surrounds the second sensor element 25. Accordingly, the first sensor receiver (22, see FIG. 8) is aligned with the first sensor element 24 regardless of the rotational position of the bioreactor (1, see FIG. 8). In the illustrated example, the first sensor element 24 is spaced from the second sensor element 25, but in other examples, they may be positioned in contact with each other.

In FIG. 9B, the first sensor element 24 is an annular sector centered on the rotational axis 26 of the bioreactor. The annular sector extends partially about the second sensor element 25. In the illustrated example, the first sensor element 24 extends about 90 degrees about the rotational axis 26 of the bioreactor, but in other examples, the first sensor element 24 may extend more or less than 90 degrees about the rotational axis 26 of the bioreactor, for example, about 45 degrees or about 270 degrees. Accordingly, the first sensor receiver (22, see FIG. 8) is aligned with the first sensor element 24 during a portion of the rotation of the bioreactor (1, see FIG. 8) corresponding to the first sensor element 24.

In FIG. 9C, the first portion 24a comprises a first annular sector and the second portion 24b comprises a second annular sector 24b, both centered on the rotational axis 26 of the bioreactor and spaced from each other. In the illustrated example, the first annular sector and the second annular sector each extend about 90 degrees about the rotational axis 26 of the bioreactor and are diametrically opposite each other. In other examples, the first annular sector and the second annular sector may extend through any angle and may be positioned abutting each other, or spaced. It will be appreciated that the first and second annular sectors do not have to extend about the same angle, for example, the first annular sector may extend 90 degrees about the rotational axis 26, and the second annular sector may extend 180 degrees about the rotational axis 26. Accordingly, the first sensor receiver (22, see FIG. 8) is aligned with the first sensor element 24 (either the first portion 24a or the second portion 24b) during a portion of the rotation of the bioreactor (1, see FIG. 8).

In the example of FIG. 9D, the first sensor element 24 comprises a plurality of sensor portions including a first portion 24a, a second portion 24b, a third portion 24c, and a fourth portion 24d, each sensor portion including a sensor element dot. In this example, the first sensor element 24 comprises four portions 24a-24d but in other examples, two or more portions including sensor element dots. In one example, the first sensor element 24 comprises twelve portions including sensor element dots. Each of the plurality of sensor portions 24a-24d is equally spaced from the rotational axis 26 of the bioreactor and corresponding to the spacing of the first sensor receiver (22, see FIG. 8). Accordingly, the first sensor receiver (22, see FIG. 8) is aligned with the first sensor element 24 (i.e., one of the sensor portions 24a-24d) at a plurality of rotational positions of the bioreactor (1, see FIG. 8). In the illustrated example, each of the sensor portions 24a-24d is circular, but they may be other shapes, for example, square.

It will be appreciated that the bioreactor may include a third sensor element, and the bioreactor system may include a third sensor receiver to detect a third characteristic of the cell suspension. In such examples, the third sensor element and the third sensor receiver may be spaced from the rotational axis 26 of the bioreactor by a different amount than the other sensor elements and sensor receivers. The third sensor element may be arranged in a corresponding arrangement to the first sensor elements. Accordingly, when the bioreactor is in a rotational position where the first sensor receiver 22 is aligned with the first sensor element 24, the third sensor receiver will also be aligned with the third sensor element. The first, second and third sensor receivers may be provided in a linear arrangement. Accordingly, the bioreactor could include additional sensor elements and the sensor unit could include additional sensor receivers.

Referring to FIG. 8, in other examples, a sensor element 37 may be positioned on the internal surface of the side wall 6 of the container 2. In such examples, the side wall 6 may include a sensor window. In such examples, a sensor receiver 38 is positioned adjacent to, or in contact with, the exterior surface of the side wall 6. The sensor element 37 either extends about the circumference of the side wall 6 or comprises a plurality of sectors or dots spaced about the circumference of the side wall 6, so that the sensor element 37 would be aligned with the sensor receiver 38 in at least two rotational positions of the bioreactor. The sensor receiver 38 is connected to a sensor meter as described above with reference to sensor receivers 22, 23. The sensor element 37 and sensor receiver 38 may be provided instead of, or in addition to, the sensor elements 24, 25 and sensor receivers 22, 23.

Throughout the description and claims of this specification, the words “comprise” and “contain” and variations of them mean “including but not limited to,” and they are not intended to (and do not) exclude other components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. A bioreactor for cell culturing, the bioreactor comprising:
a container comprising a base and a side wall defining an internal volume for holding a cell suspension, the container being rotatable during use about a rotational axis, and
a sensor element disposed on an internal surface of the container for interaction with a sensor receiver positioned externally of the container and operable to interact with the sensor element to detect a characteristic of the cell suspension, and
wherein the sensor element is offset from the rotational axis of the bioreactor and arranged to align with the sensor receiver in at least two rotational positions of the bioreactor during use.

2. The bioreactor of claim 1, wherein the sensor element is disposed on the base.

3. The bioreactor of claim 1, wherein the sensor element comprises an annulus centered on the rotational axis of the bioreactor.

4. The bioreactor of claim 1, wherein the sensor element comprises a plurality of discrete portions.

5. The bioreactor of claim 1, wherein the sensor element or at least one of the discrete portions of the sensor element comprises an annular sector.

6. The bioreactor of claim 1, wherein the sensor element comprises a dot.

7. The bioreactor of claim 1, wherein the bioreactor comprises a second sensor element disposed on an internal surface of the container, the second sensor element being aligned with the rotational axis of the bioreactor and arranged to align with a second sensor receiver positioned externally of the container and operable to interact with the second sensor element to detect a characteristic of the cell suspension.

8. The bioreactor of claim 1, wherein the sensor element is adhered to the internal surface of the container.

9. The bioreactor of claim 1, wherein the sensor element comprises a sensor chosen from an oxygen-sensitive coating and a pH-sensitive coating.

10. The bioreactor of claim 1, wherein the side wall comprises a compressible side wall including a bellows wall.

11. The bioreactor of claim 1, further comprising an interface plate attached to the side wall opposite to the base, the interface plate comprising one or more ports configured for at least one of adding fluid to and extracting fluid from the container.

12. A bioreactor system for cell culturing, the bioreactor system comprising:
a bioreactor comprising:
a container comprising a base and a side wall defining an internal volume for holding a cell suspension, the container being rotatable during use about a rotational axis, and
a sensor element disposed on an internal surface of the container for interaction with a sensor receiver positioned externally of the container and operable to interact with the sensor element to detect a characteristic of the cell suspension, and
wherein the sensor element is offset from the rotational axis of the bioreactor and arranged to align with the sensor receiver in at least two rotational positions of the bioreactor during use; and
a housing adapted to support the bioreactor with the bioreactor being rotatable relative to the housing about the rotational axis.

13. The bioreactor system of claim 12, wherein the bioreactor comprises an interface plate attached to the side wall opposite to the base, and wherein the housing comprises a bioreactor receiving portion adapted to support the interface plate with the side wall and base suspended below the interface plate.

14. The bioreactor system of claim 13, wherein the bioreactor receiving portion comprises an actuator operable to rotate the bioreactor.

15. The bioreactor system of claim 12, further comprises a sensor receiver positionable externally of the base and arranged to be aligned with the sensor element in at least two rotational positions of the bioreactor relative to the housing.

16. The bioreactor system of claim 15, wherein the sensor receiver comprises an optical receiver, including an optical fiber, and wherein the bioreactor system further comprises a sensor meter arranged to receive an optical signal from the optical receiver.

17. The bioreactor system of claim 15, wherein the sensor receiver is movably mounted and movable to an operational position in which the sensor receiver is in a position chosen from contact with, or adjacent to, the container of the bioreactor proximal to the sensor element.

18. The bioreactor system of claim 17, further comprising an actuator operable to move the sensor receiver into the operational position.

19. The bioreactor system of claim 17, wherein the side wall of the bioreactor comprises a compressible side wall including a bellows wall, and wherein the bioreactor system further comprises an agitator operable to engage the base of the bioreactor.

20. The bioreactor system of claim 19, wherein the agitator is operable to perform at least one of compress the container of the bioreactor and tilt the base of the bioreactor.

21. The bioreactor of claim 19, wherein the sensor receiver is mounted to the agitator, the sensor receiver configured to be in the operational position when the agitator engages the base of the bioreactor.

22. The bioreactor of claim 19, wherein the agitator is configured to couple to the base of the bioreactor.

23. The bioreactor of claim 22, wherein the agitator is configured to decouple from the base to permit rotation of the bioreactor relative to the housing.

24. A method of culturing cells in a bioreactor system of, the method comprising:
loading a bioreactor of the bioreactor system into a housing of the bioreactor system, the housing adapted to support the bioreactor with the bioreactor being rotatable relative to the housing about the rotational axis, the bioreactor comprising:
a container comprising a base and a side wall defining an internal volume for holding a cell suspension, the container being rotatable during use about a rotational axis, and
a sensor element disposed on an internal surface of the container for interaction with a sensor receiver positioned externally of the container and operable to interact with the sensor element to detect a characteristic of the cell suspension, and
wherein the sensor element is offset from the rotational axis of the bioreactor and arranged to align with the sensor receiver in at least two rotational positions of the bioreactor during use;
providing a cell suspension in the container of the bioreactor;
rotating the bioreactor; and
sensing a characteristic of the cell suspension by the sensor element and sensor receiver in at least two rotational positions of the bioreactor.

25. The method of claim 24, further comprising agitating the cell suspension in the container.
